(19) United States
(12) Patent Application Publication
(10) Pub. No.: US 2010/0056493 A1
(43) Pub. Date: Mar. 4, 2010

(54) MODIFIED RELEASE PHARMACEUTICAL COMPOSITION AND A PROCESS OF MAKING THE SAME

(76) Inventors: Rajesh Jain, New Delhi (IN); Kour Chand Jindal, New Delhi (IN); Sanjay Boldhane, New Delhi (IN)

Correspondence Address: LADAS & PARRY LLP, 26 WEST 61ST STREET, NEW YORK, NY, 10023, US

(21) Appl. No.: 12/522,185

(22) PCT Filed: Jan. 24, 2008
(86) PCT No.: PCT/IN08/00046

§ 371 Date: Oct. 29, 2009

(30) Foreign Application Priority Data
Jan. 25, 2007 (IN) 164/DEL/2007

(51) Int. Cl. A61K 31/554; A61K 31/5377

(52) U.S. Cl. 514/211.13; 514/233.5

(57) ABSTRACT

The present invention refers to a modified release pharmaceutical composition comprising an in-situ gelling agent (=0.5 % w/w) and a gellation facilitating agent (e.g. calcium sulfate) in an amount of 2-17.5 % w/w. Additionally, the composition contains a release rate controlling polymer such as an acrylate and optionally a pH independent rate controlling polymer such as HPMC. A preferred active agent is mycophenolate mofetil. A process of making the above described composition is also disclosed.

### FIELD OF THE INVENTION

The present invention relates to novel modified release pharmaceutical compositions comprising at least one pharmaceutically active agent(s) having a pH dependent solubility, at least one release rate controlling polymer(s) that predominantly controls the release of active agent(s) in acidic environment, a release rate modifying system that controls the release of active agent(s) in both acidic and basic environments, and optionally one or more other pharmaceutically acceptable excipient(s). The present invention also describes process for preparation of such compositions and method of using such compositions. The modified release compositions of the present invention are useful in providing prophylactically or therapeutically effective levels of active agent(s) for extended time period.

### BACKGROUND OF THE INVENTION

Many medical conditions are best treated by administration of a pharmaceutical in such a way as to modify its action over an extended period of time. Modified release dosage forms have been used with various types of pharmaceuticals such as anti-hypertensive, anti-arrhythmic, and the like. Modified release form means a formulation which releases the drug not immediately, e.g. after disintegration or in case of enteric-coating, i.e. gastro-resistant coating, after stomach passage, but offers a sustained, retard, continuous, gradual, prolonged or pulsatile release and therefore alters drug plasma levels distinctively versus an immediate release formulation. More specifically, the term “modified release formulation” as used herein refers to a formulation wherein the active agent is released and provided for absorption over a longer period of time than from a conventional dosage form, i.e. to a formulation which provides a modified release profile of the active agent contained therein.

Further, modified release compositions containing pharmaceutical medicaments or other active ingredients are designed to contain higher concentrations of an active compound and are prepared in such a manner as to affect sustained or slow release of the compound into the gastrointestinal digestive tract of humans or animals over an extended period of time. Well-absorbed oral sustained or slow release therapeutic drug dosage forms have inherent advantages over conventional, immediate release dosage forms. A less frequent dosing of a medicament, as is required by a sustained release dosage form, increases the resultant patient regime compliance, provides a more sustained drug blood level response, and effects therapeutic action with less ingestion of a drug, thereby mitigating many potential side effects. By providing a slow and steady release of a medicament over time, absorbed drug concentration spikes are mitigated or eliminated by affecting a smoother and more sustained blood level response.

However, whichever method of controlled release is utilized in the pharmaceutical formulation, such as the diffusion of the active ingredient through the coating, erosion of the coating through which the active ingredient passes, diffusion of the active ingredient from a monolithic device, to name a few, the controlled release formulation is required to meet certain criteria. Most importantly, it should result in a uniform and constant dissolution of the active ingredient from the pharmaceutical formulation to be effective for an extended period of time. It is also important that such a formulation be simple to make and that the manufacturing process be reproducible and be useful with a number of different drugs.

Mycophenolic acid, also referred to herein as MPA, was first isolated in 1896, and has been extensively investigated as a pharmaceutical of potential commercial interest. It is known to have anti-tumor, anti-viral, immunosuppressive, anti-psoriatic, and anti-inflammatory activity [see e.g. W. A. Lee et al, Pharmaceutical Research (1990), 7, p. 161-166 and references cited therein]. Publications have appeared on MPA as an anti-cancer agent by Lilly scientists, see e.g. M. J. Sweeney et al., Cancer Research (1972), 32, 1795-1802, and by ICI scientists, see e.g. GB 1,157,099 and 1,203,328 and as an immunosuppressant agent see e.g. A. Mitsui et al. J. Antibiotics (1969) 22, p. 358-363. In the above-mentioned article by W. A. Lee et al it is stated that attempts have been made to increase the bio-availability or specificity of MPA by making derivatives. The poor bioavailability of the acid was thought to be caused by undetermined factors such as drug complexation in the gastro-intestinal lumen, a narrow absorption window, metabolism before absorption etc. The preparation of the morpholinoethyl ester, also known as mycophenolate mofetil (sometimes referred to herein as MMF), was described which had considerably higher bioavailability than MPA (100% for MMF and 43% for MPA). This derivative inhibits the creation of guanosine nucleotides, one of the building blocks of DNA and RNA and has been introduced commercially as an immunosuppressant for the treatment or prevention of organ or tissue transplant rejection, at daily dosages of from about 200 mg to about 3 grams p.o, e.g. about 2 g p.o. The safety and efficacy of mycophenolate sodium compared to MMF were evaluated in a 12-month, double-blind, randomized, multicenter, parallel group study of 423 de novo kidney allograft recipients. Patients were randomized to receive either 720 mg of mycophenolic acid as mycophenolate sodium twice daily (n=213) or 1000 mg of MMF twice daily (n=210). The overall incidence of efficacy failure (universally defined throughout all studies discussed, unless otherwise noted, as biopsy-proven acute rejection [BPAR], graft loss, death, or loss to follow-up) observed in the mycophenolic acid as mycophenolate sodium and MMF groups were comparable at 6 months (28.2% and 28.1%; P=NS). BPAR rates were similar at 22.5% for mycophenolate sodium and 24.3% for MMF at 12 months (P=NS). The incidence of graft loss, patient death, and reported adverse events were similar in both groups. The incidence of GI adverse events was 78.4% with mycophenolate sodium and 78.1% with MMF (P=NS). The frequency of dosage reductions, discontinuation, or temporary interruptions of therapy secondary to GI toxicities were comparable (13.1% for mycophenolate sodium versus 17.6% for MMF; P=NS). Infection rates were similar in both groups. The authors concluded that mycophenolate sodium is therapeutically equivalent to MMF at equimolar MPA doses. 769.4 mg of mycophenolate sodium contains equimolar amounts of MPA compared with 1000 mg of MMF [Progress in Transplantation; June 2004; Gabardi, S et al].

Quetiapine fumarate is an antipsychotic drug of the dibenzothiazepine class; chemical name 2-[2-(4-dibenzo[b,f][1,4]thiazepin-11-yl-1-piperazinyl)ethoxy]-ethanol fumarate. Quetiapine acts as an antagonist at several neurotransmitter receptors including the dopamine D[sub]1 and D[sub]2 receptors, the serotonin 5HT[sub]A1 and 5HT[sub]2 receptors, the histamine H[sub]1 receptor, and adrenergic [small alpha, Greek][sub]1 and [small alpha, Greek][sub]2 receptors. Quetiapine is thought to exert its antipsychotic effects primarily via antagonism of the dopamine D2 receptor and the serotonin 5HT2 receptor. Quetiapine is currently formulated as 25 mg, 100 mg, 200 mg, and 300 mg tablets for twice a day or three times per day administration.

U.S. Pat. No. 4,753,935 describes morpholinoethylesters of mycophenolic acid i.e. mycophenolate mofetil. The said compound is useful as immunosuppressive agents, anti-inflammatory agents, anti-tumor agents, anti-viral agents, and anti-psoriatic agents. Mycophenolate mofetil is commercially sold in the US and elsewhere under the brand name CellCept®. U.S. Pat. No. 6,025,391 describes a pharmaceutical composition comprising a mycophenolate salt, the composition being adapted to prevent release of the mycophenolate salt in the stomach and to release the mycophenolate salt in the upper part of the intestinal tract. However the major limitation of formulating such a composition of mycophenolate is that although the enteric coat prevents release of the drug in the stomach to prevent associated side effects, the proper and complete absorption and/or the desired absorption pattern of the drug may not be achieved since the drug is not getting absorbed from the entire GIT, but instead restricted to absorption from only the intestine.

PCT Publication No. WO9929305 pertains to a tablet for sustained release of a drug comprising an effective amount of a drug to be released at a controlled rate and a sustained release formulation, said sustained release formulation comprising at least three different types polymers including a pH dependent gelling polymer, a pH independent gelling polymer and an enteric polymer. PCT Publication No. WO2006024479 discloses a composition comprising mycophenolic acid, a salt or a prodrug thereof in a modified release form. PCT Publication No. WO200122940 describes a sustained release oral solid dosage form comprising a therapeutically effective amount of a medicament having a solubility of more than about 10 g/l; a pH modifying agent; a sustained release matrix comprising a gelling agent, said gelling agent comprising a heteropolysaccharide gum and a homopolysaccharide gum capable of crosslinking said heteropolysaccharide gum when exposed to an environmental fluid, said dosage form providing a sustained release of said medicament after oral administration to human patients.

PCT Publication No. WO2004082615 describes a method for preparing an oral sustained release pharmaceutical composition in solid dosage form having a desired drug release profile, which pharmaceutical composition is prepared by mixing a drug with a sustained release carrier to retard the release of the drug from the pharmaceutical composition and a water insoluble or partially water insoluble cellulose to enhance the ability of the pharmaceutical composition to form the solid dosage form, resulting in a pharmaceutical composition having a drug release profile exhibiting a faster release than that of the desired drug release profile, the improvement comprising adding to the pharmaceutical composition an effective amount of a maltodextrin to retard the rate of release of the drug in the sustained release pharmaceutical composition to the desired drug release profile when placed in aqueous system, the weight ratio of the maltodextrin to the water insoluble or partially water insoluble cellulose that is added to enhance tableting ranging from about 1:50 to about 50:1.

PCT Publication No. WO2002058676 discloses a pharmaceutical composition comprising at least one pharmaceutically active agent that is pH dependent, at least one non-pH dependent sustained release agent; and at least one pH dependent agent that increases the rate of release of said at least one pharmaceutically active agent from the tablet at a pH in excess of 5. U.S. Pat. No. 4,968,508 describes a sustained release matrix formulation in tablet unit dosage from comprising from about 0.1% by weight to about 90% by weight of cefaclor, from about 5% by weight to about 29% by weight of a hydrophilic polymer, and from about 0.5% by weight to about 25% by weight of an acrylic polymer which dissolves at a pH in the range of about 5.0 to about 7.4, with the proviso that the total weight of the hydrophilic polymer and said acrylic polymer is less than 30% by weight of the formulation. U.S. Pat. No. 6,726,930, U.S. Pat. No. 6,136,343, U.S. Pat. No. 5,846,563, U.S. Pat. No. 5,667,801, U.S. Pat. No. 5,554,387, U.S. Pat. No. 5,773,025, U.S. Pat. No. 6,048,548, U.S. Pat. No. 6,245,356, U.S. Pat. No. 6,709,677, U.S. Pat. No. 5,455,046, U.S. Pat. No. 5,512,297, U.S. Pat. No. 5,662,933 and U.S. Pat. No. 5,958,456 disclose a sustained release oral solid dosage form comprising an effective amount of a medicament having a solubility of less than about 10 g/l to render a therapeutic effect; a sustained release excipient comprising a gelling agent comprising a heteropolysaccharide gum and a homopolysaccharide gum capable of cross-linking said heteropolysaccharide gum when exposed to an environmental fluid, an inert pharmaceutical diluent selected from the group consisting of monosaccharide, a disaccharide, a polyhydric alcohol, and mixtures thereof, and a pharmaceutically acceptable cationic cross-linking agent capable of crosslinking with said gelling agent and increasing the gel strength when the dosage form is exposed to an enviromental fluid; said dosage form providing a sustained release of said medicament when exposed to an environmental fluid.

Several attempts to provide dosage forms for delivery of active agents for extended periods of time have been described previously. However, there still exists a need to develop effective modified release dosage form compositions particularly comprising drugs having pH dependent solubility with reduced side effects which can provide sustained delivery of active agent, that are easier to manufacture, and involves a low formulation cost. However, formulating the drugs having pH dependent solubility such as for example, weakly basic drugs into a modified release dosage form presents a number of problems. While these drugs have relatively good solubility at gastric pH, they have a relatively poor solubility at intestinal pH. The present invention overcomes the solubility issues of these drugs in the GIT while the dosage form moves from a low pH gastric environment to a higher pH intestinal environment by describing novel compositions that provide a sustained drug release over the desired period of time to achieve the desired concentration of drug in the blood. The present invention provides such novel modified release compositions.

### SUMMARY OF THE INVENTION

It is an objective of the present invention to provide novel modified release pharmaceutical compositions comprising at least one pharmaceutically active agent(s) having a pH dependent solubility, at least one release rate controlling polymer(s) that predominantly controls the release of active agent(s) in acidic environment, a release rate modifying system that controls the release of active agent(s) in both acidic and basic environments, and optionally one or more other pharmaceutically acceptable excipient(s).

It is further an objective of the present invention to provide novel modified release pharmaceutical composition comprising at least one pharmaceutically active agent(s) having a pH dependent solubility, at least one release rate controlling polymer(s) that predominantly controls the release of active agent(s) in acidic environment, a release rate modifying system that controls the release of active agent(s) in both acidic and basic environments, wherein the release rate modifying system comprises of a combination of at least one in-situ gelling agent(s), at least one gelation facilitating agent(s) and optionally at least one pH independent rate controlling polymer(s), optionally with one or more other pharmaceutically acceptable excipient(s).

It is further an objective of the present invention to provide a modified release pharmaceutical compositions comprising at least one pharmaceutically active agent(s) having a pH dependent solubility, a release rate controlling polymer that predominantly controls the release of active agent(s) in acidic environment, a release rate modifying system that controls the release of active agent(s) in both acidic and basic environments consisting a combination of at least one in-situ gelling agent(s), at least one gelation facilitating agent(s) and at least one pH independent rate controlling polymer(s), and optionally one or more other pharmaceutically acceptable excipient(s).

It is further an objective of the present invention to provide novel modified release pharmaceutical composition comprising at least one pharmaceutically active agent(s) or its salts, polymorphs, solvates, hydrates, analogues, enantiomers, tautomeric forms, derivatives or mixtures thereof as active agent having a pH dependent solubility, either alone or in combination with other active agent(s), at least one release rate controlling polymer(s) that predominantly controls the release of active agent(s) in acidic environment, a release rate modifying system that controls the release of active agent(s) in both acidic and basic environments, wherein the release rate modifying system comprises of a combination of at least one in-situ gelling agent(s), at least one gelation facilitating agent(s) and at least one pH independent rate controlling polymer(s), optionally with other pharmaceutically acceptable excipients.

It is also an objective of the present invention to provide novel modified release pharmaceutical compositions comprising quetiapine or mycophenolate or salts, polymorphs, solvates, hydrates, analogues, enantiomers, tautomeric forms, or derivatives thereof as active agent(s) having a pH dependent solubility, at least one release rate controlling polymer(s) that predominantly controls the release of active agent(s) in acidic environment, a release rate modifying system that controls the release of active agent(s) in both acidic and basic environments, and optionally other pharmaceutically acceptable excipients.

It is another objective of the present invention to provide process for preparation of such composition which comprises of the following steps:
i) mixing the active agent(s) with release rate controlling polymer(s) and release rate modifying system,
ii) optionally adding one or more pharmaceutically acceptable excipient(s), and
iii) formulating the mixture into a suitable dosage form.

It is a further objective of the present invention to provide process for the preparation of such novel composition which comprises of the following steps:
i) mixing the active agent(s) with one or more pharmaceutically acceptable excipient(s) and granulating with release rate controlling polymer(s),
ii) mixing the granules of step (i) with the release rate modifying system,
iii) optionally adding one or more pharmaceutically acceptable excipient(s), and
iv) formulating the mixture into a suitable dosage form.

It is yet another objective of the present invention to provide a method of using such composition which comprises administering to a subject in need thereof an effective amount of the composition.

The novel compositions of the present invention are particularly useful for active agents that are absorbed throughout the GIT and thus require appreciable release in both acidic and basic pH environments such as weekly basic drugs and weekly acidic drugs. The novel compositions of the present invention provide effective prophylactic or therapeutic concentrations of active agent(s) for extended periods of time.

### DETAILED DESCRIPTION OF THE INVENTION

The present invention provides novel modified release pharmaceutical compositions comprising at least one pharmaceutically active agent(s) having a pH dependent solubility, at least one release rate controlling polymer(s) that predominantly controls the release of active agent(s) in acidic environment, a release rate modifying system that controls the release of active agent(s) in both acidic and basic environments, and optionally one or more other pharmaceutically acceptable excipient(s). According to the present invention, the active agent(s) exhibiting pH dependent solubility is a compound which is soluble at acidic pH but comparatively less soluble or insoluble at near neutral/alkaline pH, or a compound which is comparatively less soluble or insoluble at acidic pH but soluble at near neutral/alkaline pH.

In an embodiment of the present invention, the release rate modifying system comprises of a combination of at least one in-situ gelling agent(s), at least one gelation facilitating agent(s) and at least one pH independent rate controlling polymer(s), optionally with one or more other pharmaceutically acceptable excipient(s).

In an embodiment, the present invention is preferably useful for active agent(s) that has a pH dependent solubility; preferably for active agent(s) that have an appreciable release in stomach i.e. acidic pH environment but a poor release in the intestine i.e. basic pH environment. The composition of the present invention is particularly useful for active agent(s) that are absorbed throughout the gastro-intestinal tract (GIT) and thus require appreciable release in both acidic and basic pH environments such as weekly basic drugs e.g. Mycophenolate mofetil (pH=7.2) & weekly acidic drugs e.g. Quetiapine fumarate (pH=5.4).

In another embodiment of the present invention, the release rate controlling polymer(s) is present in an amount of not less than about 1.5% preferably not less than about 3% by weight of the composition. In another embodiment, the release rate modifying system comprises at least one in-situ gelling agent(s) in an amount of not less than about 2% by weight of the composition, gelation facilitating agent(s) in an amount of not less than about 0.5% of the composition and pH independent rate controlling polymer(s) in an amount of not less than about 2% by weight of the composition.

In a preferred embodiment, the present invention provides novel modified release pharmaceutical composition wherein the said system releases the active agent(s) predominantly by erosion mechanism or combination of erosion and diffusion mechanisms, preferably without any substantial deformation of shape of the dosage form, and which provides therapeutic concentrations of active agent(s) for extended periods of time.

In another embodiment, the novel modified release pharmaceutical compositions of the present invention is intended to reduce the adverse effects or side effects of the active agent(s) by controlling the peak plasma concentration (Cₘₐₓ) such that the concentration of the active agent(s) are substantially below the toxic levels but above the desired effective levels at any point of time. Also the steady state concentrations of the active agent(s) do not exhibit substantial fluctuations. The reduced incidence of side effects is thus intended to improve patient compliance with the therapy. In an embodiment, the novel compositions of the present invention releases the active agent preferably for a period of about 8-24 hours, optionally having an initial lag time wherein only 0% to about 15% of active agent(s) is released, followed by a sustained release of active agent(s). The system preferably used for controlling release rate of the active agent(s) in the present invention comprises at least one release rate controlling polymer(s) that predominantly controls the release of active agent(s) in acidic environment and a release rate modifying system that controls the release of active agent(s) in both acidic and basic environments.

The composition of the present invention is unique because the presence of polymer controlling the release of active agent(s) in acidic environment along with in-situ gelling agent(s) and gelation facilitating agent(s) present in the release rate modifying system contributes substantially towards the control of initial rapid drug release in acidic environment and facilitation of complete drug release in intestinal environment, wherein the release rate modifying system controlling the release of active agent(s) in both acidic and basic environments enhances the intactness of the dosage form, controls the rate of erosion of the dosage form and ensures the sustained release behavior of the dosage form. Furthermore, increase in the viscosity of the system due to in-situ gelling agent(s) and gelation facilitating agent(s) directly affects the extended release characteristics of the oral dosage form.

Further, it has been surprisingly found that in-situ gelling agent(s) and gelation facilitating agent(s) together form a pH dependent water-insoluble gel or gel-like structure that controls the initial drug release in acidic medium and also to some extent in small intestine but facilitate the complete drug release in large intestine due to pH dependent nature as well as enzymatic degradation of gel or gel-like structure.

The active agent of the present invention is selected from but not limited to a group comprising cardiovascular drugs, respiratory drugs, sympathomimetic drugs, cholinomimetic drugs, adrenergic agonists, adrenergic antagonists, analgesic/antipyretics, anesthetics, antiasthamatics, antibiotics, antidepressants, antidiabetics, antifungal agents, antihypertensive agents, anti-inflammatory agents, antineoplastics, antianxiety agents, antipsychotics, immunosuppressants, antimigraine agents, sedatives/hypnotics, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics useful for calcium regulation, antibacterial agents, antiviral agents, antimicrobials, anti-infectives, bronchodilators, hormones, hypoglycemic agents, hypolipidemic agents, proteins, nucleic acids, agents useful for erythropoiesis stimulation, antiulcer/antireflux agents, antinauseants/antiemetics, oil-soluble vitamins, and their pharmaceutically acceptable salts, esters, amides, polymorphs, solvates, hydrates, analogues, enantiomers, tautomeric forms or mixtures thereof, used either alone or in combination thereof.

Preferably the active agent of the present invention is an immunosuppressant selected from but not limited to a group comprising cyclosporin, tacrolimus (FK506), sirolimus (rapamycin), methotrexate, ABT578, AP23573, AP23464, AP23675, AP23841, TAFA-93, biolimus-7 or biolimus-9, mycophenolate, everolimus, azathiprine, steroids and NOX-100 or pharmaceutically acceptable salts, hydrates, polymorphs, esters, and derivatives thereof, used either alone or in combination thereof. More preferably the active agent of the present invention is selected from but not limited to a group comprising guanfacine, anagrelide, guanethidine, guanadrel, reserpine, propanolol, metoprolol, atenolol, verapamil, timolol, erythromycin, clonidine, chlorpheniramine, bromopheniramine, quetiapine, diltiazem, scopolamine, mycophenolate, and a glucocorticoid, and pharmaceutically acceptable salts, hydrates, polymorphs, esters, and derivatives thereof, used either alone or in combination thereof.

In an embodiment, the active agent(s) which is an immunosuppressant is preferably mycophenolate or its salts, polymorphs, solvates, hydrates, analogues, enantiomers,. tautomeric forms, derivatives, or mixtures thereof. In another embodiment, the active agent(s) used in the present invention is preferably an. antipsychotic drug such as quetiapine or its salts, polymorphs, solvates, hydrates, analogues, enantiomers, tautomeric forms, derivatives, or mixtures thereof.

In an embodiment of the present invention, the release rate controlling polymer(s) is selected from but not limited to group comprising copolymers of acrylate polymers with amino substituents; acrylic acid esters; polyacrylamides; phthalate derivatives (i.e., compounds with covalently attached phthalate moieties) such as acid phthalates of carbohydrates, amylose acetate phthalate, cellulose acetate phthalate, other cellulose ester phthalates, cellulose ether phthalates, hydroxypropyl cellulose phthalate, hydroxypropyl ethylcellulose phthalate, hydroxypropyl methylcellulose phthalate, methylcellulose phthalate, polyvinyl acetate phthalate, polyvinyl acetate hydrogen phthalate, sodium cellulose acetate phthalate, starch acid phthalate, styrene maleic acid dibutyl phthalate copolymer, styrene-maleic acid polyvinyl acetate phthalate copolymer; styrene and maleic acid copolymers; formalized gelatin, gluten; shellac; salol; keratin; ammoniated shellac; benzophenyl salicylate; cellulose acetate trimellitate; cellulose acetate blended with shellac; hydroxypropyl methylcellulose acetate succinate; oxidized cellulose; polyacrylic acid derivatives such as acrylic acid and acrylic ester copolymers; methacrylic acid and esters; cationic polymer with a dimethylaminoethyl ammonium group; anionic copolymer based on methyl acrylate; methyl methacrylate and methacrylic acid thereof such as poly(methacrylic acid, methyl methacrylate) 1:1 (e.g. Eudragit® L100), poly(methyacrylic acid, ethyl acrylate) 1:1 (e.g. Eudragit® L100-55, Eudragit® L30D-55), poly(methacrylic acid, methyl methacrylate) 1:2 (e.g. Eudragit® S100, Eudragit® S12.5P, Eudragit(® FS30D); vinyl acetate; crotonic acid copolymers and the like; and mixtures thereof.

Preferably the release rate controlling polymer(s) may be selected from cellulose acetate phthalate, hydroxypropyl methylcellulose phthalate, polvinyl acetate phthalate, acrylic acid or methacrylic acid copolymers, cellulose acetate trimellitate, hydroxypropyl methylcellulose acetate, succinate, shellac, and zein. Preferably the release rate controlling polymer(s) is present in an amount of not less than about 1.5% w/w of the composition, more preferably in an amount of about 3-80% w/w of the composition.

In another embodiment of the present invention, the release rate modifying system comprises of a combination of at least one in-situ gelling agent(s), at least one gelation facilitating agent(s) and at least one pH independent rate controlling polymer(s).

In an embodiment of the present invention, the in-situ gelling agent is selected from but not limited to a group comprising locust bean gum, xanthan gum, tragacanth, xylan, arabinogalactan, agar, gellan gum, scleroglucan, guar gum, apricot gum (Prunus armeniaca, L.), alginate, carrageenan, pectin (Genu®), acacia gum, dextran, and gum arabic and the like or mixtures thereof. In a preferred embodiment, pectin is used as the in-situ gelling agent. Preferably the in-situ gelling agent is present in an amount of not less than about 0.5% w/w of the composition.

In an embodiment of the present invention, the gelation facilitating agent is selected from but not limited to a group comprising calcium sulfate, calcium chloride, aluminium chloride, magnesium chloride, calcium lactate, calcium citrate, magnesium citrate and magnesium sulfate. Preferably the gelation facilitating agent is divalent or trivalent cation salt. More preferably, calcium sulfate is used as gelation facilitating agent and present in an amount not less than about 0.5% w/w of the composition, most preferably about 2-17.5% w/w of the composition. In an embodiment, the gelation facilitating agent acts as a crosslinking agent.

In an embodiment of the present invention, the pH independent polymer is selected from but not limited to a group comprising alkyl celluloses such as methyl cellulose, hydroxyalkyl alkyl celluloses such as hydroxypropyl methyl cellulose (HPMC, HPMC® K100M CR, Methocel®), hydroxy alkyl celluloses such as hydroxypropyl cellulose (HPC, Klucel®) and hydroxy ethyl cellulose (HEC, Natrosol®t), polyethylene glycols (PEG®, Lutrol®), copolymers of ethylene oxide with propylene oxide (Poloxamer®), gelatin, polyvinylpyrrolidones (PVP, Kollidon®), vinylpyrrolidones, vinyl acetates, polyvinylimidazoles, polyvinylpyridine N-oxides, copolymers of vinylpyrrolidone with long-chained alpha-olefins, copolymers of vinylpyrrolidone with vinylimidazole, poly(vinylpyrrolidone/dimethylaminoethyl methacrylates), copolymers of vinylpyrrolidone/dimethylaminopropyl methacrylamides, copolymers of vinylpyrrolidone/dimethylaminopropyl acrylamides, quaternised copolymers of vinylpyrrolidones and dimethylaminoethyl methacrylates, terpolymers of vinylcaprolactam/vinylpyrrolidone/dimethylaminoethyl methacrylates, copolymers of vinylpyrrolidone and methacrylamidopropyl-trimethylammonium chloride, terpolymers of caprolactam/vinylpyrrolidone/dimethylaminoethyl methacrylates, copolymers of styrene and acrylic acid, polycarboxylic acids, polyvinyl alcohols (PVA, Mowiol®), hydrolysed polyvinyl acetate, polysaccharide gums, both natural and modified (semi-synthetic), including but not limited to xanthan gum, veegum, agar, guar gum, locust bean gum, gum arabic, okra gum, alginic acid, other alginates (e.g. sodium alginate, propyleneglycol alginate), benitonite, arabinoglactin, pectin, tragacanth, scleroglucan, dextran, amylose, amylopectin, dextrin, and the like, or mixtures thereof

Preferably the pH independent polymer is one or more of hydroxyalkyl alkyl celluloses, more preferably hydroxypropyl methylcellulose. Preferably the pH independent polymer is present in an amount of not less than about 1% w/w of the composition, more preferably about 2-40% w/w of the composition.

In a preferred embodiment of the present invention, the ratio of the in-situ gelling agent(s) and the gelation facilitating agent(s) is about 1:10 to about 10:1, preferably about 1:5 to about 5:1 by weight of the composition.

In an embodiment, the composition of the present invention additionally comprises one or more pharmaceutically acceptable excipients selected from but not limited to a group comprising diluent and a solvent. In an embodiment of the present invention, the diluent is selected from but not limited to a group comprising microcrystalline cellulose, lactose, starch, dibasic calcium phosphate, saccharides, and mixtures of the foregoing. Examples of diluents include microcrystalline celluloses (Avicel®); lactose such as lactose monohydrate, lactose anhydrous (Pharmatose®), and lactose spray dried forms; dibasic calcium phosphate (Emcompress®); mannitol (Pearlitol®); starch; sorbitol; sucrose; glucose; cyclodextrins; and the like or mixtures thereof. In the present invention, the solvent used is selected from but not limited to a group comprising alcohols such as methanol, ethanol, propanol, isopropyl alcohol, butanol, monomethoxyethanol, ethylene glycol monomethylether and the like; ethers such as diethyl ether, dibutyl ether, diisobutyl ether, dioxane, tetrahydrofuran, ethylene glycol and the like; aliphatic hydrocarbons such as n-hexane, cyclohexane and n-heptane; aromatic hydrocarbons such as benzene, toluene and xylene; nitriles such as acetonitrile and the like; organic acids such as acetic acid, propionic acid and the like; esters such as ethyl acetate; aliphatic halogenated hydrocarbons such as dichloromethane, dichloroethane, chloroform and the like; ketones such as acetone, methyl ketone and the like; amides such as dimethylformamide, dimethyl acetamide and the like; or mixtures thereof Among the solvents, the one having a low boiling point such as ketones e.g. acetone and alcohols e.g. ethanol is preferable. More preferably the solvent used is dichloromethane and is in quantity sufficient to dissolve or disperse the solubilizer and/or the active agent(s).

The one or more pharmaceutically acceptable excipient(s) of the present invention are selected from but not limited to a group comprising disintegrants, binders, fillers, bulking agents, anti-adherants, anti-oxidants, buffering agents, colorants, flavoring agents, coating agents, plasticizers, stabilizers, preservatives, lubricants, glidants, chelating agents, and the like known to the art used either alone or in combination thereof. Certain excipients used in the present composition can serve more than one purpose.

Suitable binders include for example starch, polyvinylpyrrolidone, hydroxypropyl methylcellulose, pregelatinised starch, hydroxypropylcellulose, or mixtures thereof. Suitable lubricants are selected from but not limited to a group comprising colloidal silicon dioxide such as Aerosil® 200, talc, stearic acid, magnesium stearate, calcium stearate, sodium stearyl fumarate, hydrogenated vegetable oil and the like, or mixtures thereof. Suitable disintegrants include for example crosslinked polyvinyl pyrrolidone, corn starch, potato starch, maize starch and modified starches, croscarmellose sodium, sodium starch glycollate, carboxymethyl cellulose calcium, or mixtures thereof.

In an embodiment, the composition of the present invention comprising at least one pharmaceutically active agent(s) having a pH dependent solubility, comprises of at least one pH dependent polymer(s), at least one water soluble in-situ gelling polymer(s), at least one cross-linking agent(s) and at least one pH independent polymer(s), forms a matrix wherein the active agent is released by diffusion into the gastric enviromnent and by erosion into the intestinal environment. The polymers used in the present invention help in modifying the release of active agent(s) in following manner: The pH dependent polymer(s) controls the initial burst release of the active agent(s) in acidic medium and prevent dose dumping that may occur due to higher solubility of drug in acidic medium. Also, the pH dependent polymer(s) facilitates erosion and in-turn drug release above pH 5.5. The in-situ gelling agent(s) forms a water insoluble gel upon cross-linking with cross-linking agent when the said polymer comes in contact with dissolved ions (of cross-linking agent) in-vivo. The formed insoluble gel controls the initial drug release in acidic medium and also to some extent in small intestinal environment but facilitates substantially complete drug release before or upon exposure of the composition to large intestinal environment due to pH dependent nature as well as enzymatic degradation of gel. The pH independent polymer(s) provides integrity to dosage form till substantially complete drug release occurs and also controls the rate of drug release.

In an embodiment of the present invention is provided a process for preparation of such composition which comprises treating the active agent(s) with release rate controlling polymer(s) and release rate modifying system, optionally adding one or more pharmaceutically acceptable excipient(s), and formulating the mixture into a suitable dosage form. In a further embodiment, the process for the preparation of the novel compositions of the present invention comprises mixing the active agent(s) with one or more pharmaceutically acceptable excipient and granulating with release rate controlling polymer(s), mixing the granules thus obtained with the release rate modifying system, optionally adding one or more pharmaceutically acceptable excipient(s), and formulating the mixture into a suitable dosage form.

In a further embodiment, the composition of the present invention is preferably formulated as a solid dosage form such as tablets/minitablets, capsules, pellets or the like, more preferably as tablets. The tablets can be prepared by either wet granulation, direct compression, or by dry compression (slugging). In a preferred embodiment of the present invention, the oral composition is prepared by wet granulation. The granulation technique is either aqueous or non-aqueous. The non-aqueous solvent used is selected from a group comprising acetone, ethanol, isopropyl alcohol and methylene chloride. In an embodiment, the compositions of the present invention are in the form of compressed tablets, moulded tablets, mini-tablets, capsules, compacts, pellets, granules and the like. The tablets may be optionally coated with a nonfunctional coating to form a nonfunctional layer. The tablets/minitablets may be optionally filed into capsules.

In yet another embodiment of the present invention is provided a method of using such novel sustained release compositions which comprises administering to a subject in need thereof an effective amount of the composition. In a further embodiment, the composition of the present invention may be useful for the management such as prophylaxis, amelioration or treatment of one or more diseases or disorders depending upon the nature and quantity of the active agent(s) used to formulate the compositions. For example, compositions comprising mycophenolate as active agent is useful in the management of anti-tumor, anti-viral, immunosuppressive, anti-psoriatic, and anti-inflammatory activity. Alternatively, compositions comprising quetiapine as active agent is useful in the management of psychosis.

In another embodiment, the novel compositions of the present invention are particularly useful for active agents that are absorbed throughout the GIT and thus require appreciable release in both acidic and basic pH environments such as weakly basic drugs and weakly acidic drugs. The novel compositions of the present invention provide effective prophylactic or therapeutic concentrations of active agent(s) for extended periods of time.

The examples given below serve to illustrate embodiments of the present invention.

However they do not intend to limit the scope of present invention.

### Examples

### Example-1

| S. No. | Ingredient | mg/tablet |
|---|---|---|
| 1. | Mycophenolate mofetil | 1011.28 |
| 2. | Lactose anhydrous | 74.25 |
| 3. | Polyethyl acrylate (Eudragit ® L30 D55) | 67.50 |
| 4. | Copovidone | 04.59 |
| 5. | Pectin (Genu ®) | 60.75 |
| 6. | Hydroxypropylmethyl cellulose | 87.75 |
| | (HPMC ® K100M CR) | |
| 7. | Calcium sulphate | 30.38 |
| 8. | Magnesium stearate | 13.50 |

Coating composition

| | | |
|---|---|---|
| 9. | Opadry ® white dispersion (in water) | q.s. |

Procedure:
i) Mycophenolate mofetil and Lactose anhydrous were passed through #40 mesh and granulated with aqueous Polyethyl acrylate dispersion containing Copovidone and dried.
ii) Pectin and Calcium sulfate were mixed together and Hydroxypropylmethyl cellulose was added thereafter and mixed well.
iii) The above granules of step (i) were mixed with blend of step (ii).
iv) The above blend of step (iii) was lubricated with Magnesium stearate and compressed into tablets.
v) The compressed tablets were coated with Opadry® white to a weight gain of 5.0%w/w.

### Example-2

| S. No. | Ingredient | mg/tablet |
|---|---|---|
| 1. | Quetiapine (as Quetiapine fumarate) | 400.00 |
| 2. | Hydroxyethylcellulose phthalate | 75.00 |
| 3. | Dibasic calcium phosphate | 49.05 |
| 4. | Microcrystalline cellulose | 37.50 |
| 5. | Xanthan gum | 37.50 |
| 6. | Sodium alginate | 60.00 |
| 7. | Calcium chloride | 18.75 |
| 8. | Magnesium stearate | 7.50 |

Procedure:
i) Quetiapine (as fumarate), Microcrystalline cellulose and Dibasic calcium phosphate were mixed together.
ii) The blend of step (i) was sifted through #40 sieve.
iii) Step (ii) blend was granulated by using Hydroxyethyl cellulose phthalate solution in acetone:ethanol (1:1).
iv) The granules of step (iii) were sifted through sieve.
v) Xanthan gum and Calcium chloride were sifted separately through #40 sieve and then mixed well with step (iv) granules.
vi) Sodium alginate was sifted through #40 sieve and was mixed well with step (v) blend.
vii) Final blend of step (vi) was lubricated with Magnesium stearate and compressed into tablets.

### Example-3

| S. No. | Ingredient | mg/tablet |
|---|---|---|
| 1. | Mycophenolate (as Mycophenolate mofetil) | 750.00 |
| 2. | Polyethyl acrylate | 120.00 |
| 3. | Anhydrous lactose | 144.00 |
| 4. | Pectin | 60.00 |
| 5. | Hydroxypropylmethyl cellulose | 84.00 |
| 6. | Calcium sulfate dihydrate | 30.00 |
| 7. | Magnesium stearate | 12.00 |

Coating composition

| | | |
|---|---|---|
| 8. | Opadry ® yellow dispersion (in water) | q.s. |

Procedure:
i) Mycophenolate mofetil and Lactose were mixed together.
ii) The blend of step (i) was sifted through #40 sieve.
iii) Step (ii) blend was granulated using Polyethyl acrylate.
iv) The granules of step (iii) were dried and sifted through sieve.
v) Pectin and Calcium sulfate dihydrate were sifted separately through #40 sieve and then mixed well, followed by mixing the blend with step (iv) granules.
vi) Hydroxypropylmethyl cellulose was sifted through #40 sieve and mixed with step (v) blend.
vii) Final blend of step (vi) was lubricated with Magnesium stearate and compressed into tablets.
viii) The tablets of step (vii) were coated with the Opadry® yellow dispersion (in water) and dried.

### Example-4

| S. No. | Ingredient | mg/tablet |
|---|---|---|
| 1. | Mycophenolate mofetil | 500.00 |
| 2. | Polyethyl acrylate | 100.00 |
| 3. | Anhydrous lactose | 96.00 |
| 4. | Pectin | 66.00 |
| 5. | Calcium sulfate dihydrate | 30.00 |
| 6. | Magnesium stearate | 8.00 |

Procedure:
i) Mycophenolate mofetil and lactose were mixed well.
ii) The blend of step (i) was sifted through #40 sieve.
iii) Step (ii) blend was granulated by using Polyethyl acrylate.
iv) The granules of step (iii) were dried and sifted through sieve.
v) Pectin and Calcium sulfate dihydrate were sifted separately through #40 sieve and then mixed well, followed by mixing the blend with step (iv) granules.
vi) Final blend of step (v) was lubricated with Magnesium stearate and compressed into tablets.

### Example-5

| S. No. | Ingredient | mg/tablet |
|---|---|---|
| 1. | Diltiazem Hydrochloride | 360.00 |
| 2. | Hydroxymethylcellulose phthalate | 65.00 |
| 3. | Mannitol | 50.00 |
| 4. | Lactose | 35.00 |
| 5. | Gellan gum | 39.50 |
| 6. | Hydroxypropyl cellulose | 58.50 |
| 7. | Calcium sulfate | 17.75 |
| 8. | Stearic acid | 7.25 |

Procedure:
i) Diltiazem, Mannitol and Lactose were mixed together.
ii) The blend of step (i) was sifted through #40 sieve.
iii) Step (ii) blend was granulated by using Hydroxymethylcellulose phthalate.
iv) The granules of step (iii) were sifted through sieve.
v) Gellan gum and Calcium sulfate were sifted separately through #40 sieve and then mixed well with step (iv) granules.
vi) Hydroxypropyl cellulose was sifted through #40 sieve and was mixed well with step (v) blend.
vii) Final blend of step (vi) was lubricated with Stearic acid and compressed into tablets.

### Example-6

| S. No. | Ingredient | mg/capsule |
|---|---|---|
| 1. | Atenolol | 100.00 |
| 2. | Cellulose acetate phthalate | 67.00 |
| 3. | Lactose | 75.00 |
| 4. | Pectin | 39.50 |
| 5. | Hydroxypropyl cellulose | 58.50 |
| 6. | Calcium sulfate | 17.75 |

Procedure:
i) Atenolol and Lactose were mixed together.
ii) The blend of step (i) was sifted through #40 sieve.
iii) Step (ii) blend was granulated by using Cellulose acetate phthalate.
iv) The granules of step (iii) were sifted through sieve.
v) Pectin and Calcium sulfate were sifted separately through #40 sieve and then mixed with step (iv) granules.
vi) Hydroxypropyl cellulose was sifted through #40 sieve and was mixed well with step (v) blend.
vii) The blend of step (vi) was sifted through #40 sieve.
viii) The granules of step (vii) were filled into hard gelatin capsules.

### Example-7

| S. No. | Ingredient | mg/capsule |
|---|---|---|
| 1. | Quetiapine (as Quetiapine fumarate) | 200.00 |
| 2. | Cellulose acetate trimellitate | 18.75 |
| 3. | Microcrystalline cellulose | 65.77 |
| 4. | Guar gum | 18.75 |
| 5. | Hydroxypropylmethyl cellulose | 26.25 |
| 6. | Calcium chloride | 9.38 |
| 7. | Talc | 3.75 |

Coating composition

| | | |
|---|---|---|
| 8. | Opadry ® yellow dispersion (in water) | q.s. |

Procedure:
i) Quetiapine fumarate and Microcrystalline cellulose were mixed well.
ii) The blend of step (i) was sifted through #40 sieve.
iii) Step (ii) blend was granulated using Cellulose acetate trimellitate.
iv) The granules of step (iii) were sifted through suitable sieve.
v) Guar gum and Calcium chloride were sifted separately through #40 sieve and then mixed well with step (iv) granules.
vi) Hydroxypropylmethyl cellulose was sifted through #40 sieve and mixed with step (v) blend.
vii) Final blend of step (vi) was lubricated with sifted Talc and compressed into minitablets.
viii) The tablets of step (vii) were coated with the Opadry(® yellow dispersion (in water) and dried.
ix) The coated minitablets of step (viii) were filed into hard gelatin capsule.

### Example-8

| S. No. | Ingredient | mg/capsule |
|---|---|---|
| 1. | Anagrelide | 1.00 |
| 2. | Hydroxypropyl methylcellulose acetate | 21.00 |
| 3. | Anhydrous lactose | 15.65 |
| 4. | Xanthan gum | 7.00 |
| 5. | Sodium alginate | 17.50 |
| 6. | Magnesium chloride | 2.10 |
| 7. | Stearic acid | 5.75 |

Coating composition

| | | |
|---|---|---|
| 8. | Opadry ® yellow dispersion (in water) | q.s. |

Procedure:
i) Anagrelide and Lactose were mixed well.
ii) The blend of step (i) was sifted through #40 sieve.
iii) Step (ii) blend was granulated using Hydroxypropyl methylcellulose acetate.
iv) The granules of step (iii) were sifted through suitable sieve.
v) Xanthan gum and Magnesium chloride were sifted separately through #40 sieve and then mixed well with step (iv) granules.
vi) Sodium alginate was sifted through #40 sieve and mixed well with step (v) blend.
vii) Final blend of step (vi) was lubricated with Stearic acid and compressed into tablets.
viii) The tablets of step (vii) were coated with the Opadry® yellow dispersion (in water) and dried.

### Example-9

| S. No. | Ingredient | mg/tablet |
|---|---|---|
| 1. | Quetiapine fumarate | 54.29 |
| 2. | Lactose anhydrous | 24.96 |
| 3. | Polyethyl acrylate (Eudragit ® L30D 55) | 5.00 |
| 4. | Polyvinyl pyrrolidone (PVP ® K30) | 0.25 |
| 5. | Pectin | 5.00 |
| 6. | Calcium Sulphate | 2.50 |
| 7. | Hydroxypropylmethyl cellulose | 7.00 |
| 8. | Magnesium stearate | 1.00 |

Procedure:.
i) Quetiapine fumarate and Lactose were mixed and sifted through #40 sieve.
ii) The blend was granulated using solution of Polyethyl acrylate and Polyvinyl pyrrolidone.
iii) The granules of step (ii) were sifted through sieve.
iv) Pectin and Calcium sulfate were sifted separately through through #40 sieve and then mixed well with step (iii) granules.
v) Hydroxypropylmethyl cellulose was sifted through #40 and mixed with step (iv) blend.
vi) The blend was lubricated with Magnesium stearate and compressed into tablets.

### Example-10

| S. No. | Ingredient | mg/tablet |
|---|---|---|
| 1. | Mycophenolate mofetil | 750.00 |
| 2. | Lactose anhydrous | 2.04 |
| 3. | Polyethyl acrylate (Eudragit ® L30 D55) | 5.00 |
| 4. | Copovidone | 0.17 |
| 5. | Pectin | 5.00 |
| 6. | Calcium Sulphate | 2.00 |
| 7. | Hydroxypropylmethyl cellulose | 7.00 |
| 8. | Magnesium stearate | 1.00 |

Procedure:
i) Mycophenolate mofetil and Lactose were mixed together.
ii) The blend of step (i) was sifted through #40 sieve.
iii) Polyethyl acrylate and Copovidone were added in purified water.
iv) Step (ii) blend was granulated by using solution of step (iii).
v) The granules of step (iv) were sifted through sieve.
vi) Pectin and Calcium sulfate were sifted separately through through #40 sieve and then mixed well with step (v) granules.
vii) Hydroxypropylmethyl cellulose was sifted through #40 sieve and was mixed well with step (vi) blend.
viii) Blend of step (vii) was lubricated with Magnesium stearate and compressed into tablets.

1. A modified release pharmaceutical compositions comprising at least one pharmaceutically active agent(s) having a pH dependent solubility, a release rate controlling polymer that predominantly controls the release of active, agent(s) in acidic environment in an amount of 3-80% w/w of the composition, a release rate modifying system that controls the release of active agent(s) in both acidic and basic environments consisting a combination of at least one in-situ gelling agent(s) in an amount of not less than about 0.5% w/w of the composition, at least one gelation facilitating agent(s) in an amount of 2-17.5% w/w of the composition and optionally at least one pH independent rate controlling polymer(s) in an amount up to 40% w/w of the composition, and optionally one or more other pharmaceutically acceptable excipient(s).

2. The composition as claimed in claim 1, wherein the active agent is an immunosuppressant selected from a group comprising cyclosporin, tacrolimus (FK506), sirolimus (rapamycin), methotrexate, ABT578, AP23573, AP23464, AP23675, AP23841, TAFA-93, biolimus-7 or biolimus-9, mycophenolate, everolimus, azathiprine, steroids, NOX-100, and pharmaceutically acceptable salts, hydrates, polymorphs, esters, derivatives thereof, used either alone or in combination thereof.

3. The composition as claimed in claim 1, wherein the active agent is selected from a group comprising guanfacine, anagrelide, guanethidine, guanadrel, reserpine, propanolol, metoprolol, atenolol, verapamil, timolol, erythromycin, clonidine, chlorpheniramine, bromopheniramine, quetiapine, diltiazem, scopolamine, glucocorticoid, and pharmaceutically acceptable salts, hydrates, polymorphs, esters, derivatives thereof, used either alone or in combination thereof.

4. The composition as claimed in claim 1, wherein the active agent is mycophenolate or quetiapine, or pharmaceutically acceptable salts, hydrates, polymorphs, esters, or derivatives thereof.

5. The composition as claimed in claim 4, wherein the active agent is mycophenolate mofetil.

6. The composition as claimed in claim 4, wherein the active agent is quetiapine fumarate.

7. The composition as claimed in claim 1, wherein the release rate controlling polymer is selected from a group comprising copolymers of acrylate polymers with amino substituents; acrylic acid esters; polyacrylamides; phthalate derivatives such as acid phthalates of carbohydrates, amylose acetate phthalate, cellulose acetate phthalate; other cellulose ester phthalates; cellulose ether phthalates; hydroxypropyl cellulose phthalate; hydroxypropyl ethylcellulose phthalate; hydroxypropyl methylcellulose phthalate; methylcellulose phthalate; polyvinyl acetate phthalate; polyvinyl acetate hydrogen phthalate; sodium cellulose acetate phthalate; starch acid phthalate; styrene maleic acid dibutyl phthalate copolymer; styrene-maleic acid polyvinyl acetate phthalate copolymer; styrene and maleic acid copolymers; formalized gelatin; gluten; shellac; salol; keratin; ammoniated shellac; benzophenyl salicylate; cellulose acetate trimellitate; cellulose acetate blended with shellac; hydroxypropyl methylcellulose acetate succinate; oxidized cellulose; polyacrylic acid derivatives; methacrylic acid and esters; cationic polymer with a dimethylaminoethyl ammonium group; anionic copolymer based on methyl acrylate; methyl methacrylate and methacrylic acid thereof; vinyl acetate; crotonic acid copolymers; and mixtures thereof.

8. The composition as claimed in claim 1, wherein the in-situ gelling agent is selected from a group comprising locust bean gum, xanthan gum, tragacanth, xylan, arabinogalactan, agar, gellan gum, scleroglucan, guar gum, apricot gum, alginate, carrageenan, pectin, acacia gum, dextran, gum arabic, and mixtures thereof.

9. The composition as claimed in claim 1, wherein the gelation facilitating agent is selected from a group comprising calcium sulfate, calcium chloride, aluminium chloride, magnesium chloride, calcium lactate, calcium citrate, magnesium citrate and magnesium sulfate.

10. The composition as claimed in claim 1, wherein the pH independent polymer is selected from a group comprising alkyl celluloses, hydroxyalkyl alkyl celluloses, hydroxy alkyl celluloses, polyethylene glycols, copolymers of ethylene oxide with propylene oxide, gelatin, polyvinylpyrrolidones, vinylpyrrolidones, vinyl acetates, polyvinylimidazoles, polyvinylpyridine-oxides, copolymers of vinylpyrrolidone with long-chained alpha-olefins, copolymers of vinylpyrrolidone with vinylimidazole, poly(vinylpyrrolidone/dimethylaminoethyl methacrylates), copolymers of vinylpyrrolidone/dimethylaminopropyl methacrylamides, copolymers of vinylpyrrolidone/dimethylaminopropyl acrylamides, quaternised copolymers of vinylpyrrolidones and dimethylaminoethyl methacrylates, terpolymers of vinylcaprolactam/vinylpyrrolidone/ dimethylaminoethyl methacrylates, copolymers of vinylpyrrolidone and methacrylamidopropyl-trimethylammonium chloride, terpolymers of caprolactam/vinylpyrrolidone/dimethylaminoethyl methacrylates, copolymers of styrene and acrylic acid, polycarboxylic acids, polyvinyl alcohols, hydrolysed polyvinyl acetate, or mixtures thereof.

11. The composition as claimed in claim 10, wherein the pH independent polymer is hydroxyalkyl alkyl cellulose.

12. The composition as claimed in claim 11, wherein the hydroxyalkyl alkyl cellulose is hydroxypropylmethyl cellulose.

13. The composition as claimed in claim 1, wherein the in-situ gelling agent and the gelation facilitating agent is in a ratio of 1:10 to 10:1.

14. The composition as claimed in claim 1, wherein the pharmaceutically acceptable excipients are selected from a group comprising disintegrants, binders, fillers, bulking agents, anti-adherants, anti-oxidants, buffering agents, colorants, flavoring agents, coating agents, plasticizers, stabilizers, preservatives, lubricants, glidants, and chelating agents used either alone or in combination thereof.

15. A process of preparation of the composition as claimed in claim 1, which comprises of the following steps:
i) mixing the active agent(s) with release rate controlling polymer(s) and release rate modifying system,
ii) optionally adding one or more pharmaceutically acceptable excipient(s), and
iii) formulating the mixture into a suitable dosage form.

16. A process for the preparation of the composition as claimed in claim 1, which comprises of the following steps:
i) mixing the active agent(s) with one or more pharmaceutically acceptable excipient(s) and granulating with release rate controlling polymer(s),
ii) mixing the granules of step (i) with the release rate modifying system,
iii) optionally adding one or more pharmaceutically acceptable excipient(s), and
iv) formulating the mixture into a suitable dosage form.

17. A method of using a composition as claimed in claim 1, which comprises administering to a subject in need thereof an effective amount of the composition for the management, prophylaxis amelioration or treatment of tumor, organ or tissue transplant rejection; psoriasis inflammatory activity or viral activity.

18. (canceled)

19. (canceled)

20. (canceled)
